(19) United States
(12) Patent Application Publication
(10) Pub. No.: US 2026/0014161 A1
(43) Pub. Date: Jan. 15, 2026

(54) Uses of JAK Inhibitors in the Management of Inflammation-Associated Depression and Central Nervous System (CNS) Pathologies

(71) Applicants: Emory University, Atlanta, GA (US); The University of Queensland

(72) Inventors: Christina Gavegnano, Atlanta, GA (US); Andrew H. Miller, Atlanta, GA (US); Susan Pereira, Atlanta, GA (US); Susannah Tye, St. Lucia (AU); Zhexing Wen, Marietta, GA (US); Emily Edwards, Decatur, GA (US); Sebastian Roa, Atlanta, GA (US)

(21) Appl. No.: 19/281,004

(22) Filed: Jul. 25, 2025

Related U.S. Application Data
(63) Continuation of application No. 18/875,105, filed on Dec. 13, 2024.
(60) Provisional application No. 63/351,456, filed on Jun. 13, 2022.

(51) Int. Cl. A61K 31/519; A61K 31/436; A61K 31/4985; A61K 31/529; A61K 31/541; A61K 31/635; A61K 45/06; A61P 25/24

(52) U.S. Cl. CPC A61K 31/519; A61K 31/436; A61K 31/4985; A61K 31/529; A61K 31/541; A61K 31/635; A61K 45/06; A61P 25/24

(57) ABSTRACT

This disclosure relates to methods of treating, preventing, or reversing inflammation-associated central nervous system (CNS) disorders or conditions comprising administering an effective amount of a Janus kinases (JAK) inhibitor such as baricitinib to a patient in need thereof. In certain embodiments, the CNS associated inflammatory disorder is depression, treatment resistant depression, fatigue, or cognitive dysfunction.

### CROSS-REFERENCE TO RELATED APPLICATIONS

This application is a continuation of U.S. patent application Ser. No. 18/875,105 filed Dec. 13, 2024, which is the National Stage of International Application No. PCT/US2023/025208 filed Jun. 13, 2023, which claims the benefit of U.S. Provisional Application No. 63/351,456 filed Jun. 13, 2022. The entirety of each of these applications is hereby incorporated by reference for all purposes

### BACKGROUND

Depression is a major health problem. The effectiveness of antidepressants is highly variable, and many patients attempt several treatment options without improvement. Signs of Treatment-Resistant Depression (TRD) include more frequent, severe, and longer episodes of depression, short moments of improvement followed by depression symptoms, anxiety, or an anxiety disorder. Thus, there is a need to identify improved therapies for depression and TRD. Both depression and TRD as well as other CNS pathologies have been associated with markers of inflammation in the blood and in the brain.

Janus kinases (JAKs) are cytoplasmic tyrosine kinases which include JAK1, JAK2, JAK3, and TYK2. The JAKs signal through a biological pathway which regulates the transcription of several genes involved in inflammation and cancer. Shawky et al. report an overview of globally approved JAK inhibitors. Pharmaceutics, 2022, 14, 1001.

Thyssen et al. report improvement in symptoms of anxiety and depression in patients with atopic dermatitis after treatment with baricitinib. J Eur Acad Dermatol Venereol, 2022, 36(2):e147-e150.

See also Gavegnano & Ribeiro, International PCT Publ. No. WO 2022/251434.

References cited herein are not an admission of prior art.

### SUMMARY

This disclosure relates to methods of treating, preventing, or reversing inflammation-associated central nervous system (CNS) disorders or conditions comprising administering an effective amount of a Janus kinases (JAK) inhibitor such as baricitinib to a patient in need thereof. In certain embodiments, the inflammation-associated CNS disorder is depression, treatment resistant depression, and related depression symptoms of apathy, suicidal ideologies, fatigue, or cognitive dysfunction.

In certain embodiments, this disclosure relates to methods of treating inflammation-associated depression comprising administering an effective amount of a JAK inhibitor such as baricitinib to a patient in need thereof. In certain embodiments, this disclosure relates to methods of treating depression comprising administering an effective amount of a JAK inhibitor such as baricitinib to a patient in need thereof. In certain embodiments, methods reported herein are contemplated to exclude patients having atopic dermatitis, rheumatoid arthritis, or alopecia.

In certain embodiments, the patient was administered an antidepressant previous to administering the JAK inhibitor/baricitinib and did not respond or becomes non-responsive to the antidepressant treatment. In certain embodiments, the patient was previously administered an antidepressant and continued to present with clinical depression. In certain embodiments, the patient was previously administered two different antidepressants and continued to present with clinical depression. In certain embodiments, baricitinib is administered in combination with another antidepressant.

In certain embodiments, this disclosure relates to methods of treating inflammation-associated symptoms of depression including fatigue and cognitive dysfunction comprising administering an effective amount of a JAK inhibitor such as baricitinib to a patient in need thereof.

In certain embodiments, this disclosure relates to methods of treating inflammation-associated schizophrenia comprising administering an effective amount of a JAK inhibitor such as baricitinib to a patient in need thereof.

In certain embodiments, this disclosure relates to methods of treating inflammation-associated dopamine disruption to improve memory/cognition, motivation, motor activity, (treat or prevent) psychomotor slowing, prevent fatigue, improve anhedonia, improve memory, improve concentration, improve cognition, prevent (reduce) sleep disruptions, and prevent suicide comprising administering an effective amount of a Janus kinases (JAK) inhibitor such as baricitinib to a patient in need thereof.

In certain embodiments, for any of the methods disclosed herein the JAK inhibitor may be administered in combination with another drug, antidepressant, antipsychotic, or anti-inflammatory agent.

### BRIEF DESCRIPTION OF THE SEVERAL VIEWS OF THE DRAWINGS

FIGS. 1A-1F show data indicating 1 uM baricitinib significantly reduces inflammation in primary monocytes and myeloid cells, a key driver of Treatment Resistant Depression and other inflammatory dysregulatory pathologies.

FIG. 1A shows data on CD69 cells.

FIG. 1B shows data on TCF-1 cells.

FIG. 1C shows data on the frequency of parent cells.

FIG. 1D shows data on CD69 cells.

FIG. 1E shows data on GzB cells.

FIG. 1F shows data on TCF-1 cells.

FIGS. 1G-1H show data indicating IL-6 induces molecular signatures of depression (dopaminergic neurons), and baricitinib significantly reverses these signatures of depression. Molecular signatures associated with IL-6 treatment in human DA neurons.

FIG. 1G shows Gene Ontology (GO) analyses showing biological pathways, cellular compartments, and molecular functions enriched in specific sets of up-regulated genes from IL-6 treated DA neurons.

FIG. 1H shows down-regulated genes from IL-6+baricitinib-treated DA neurons. This data indicates that baricitinib can reverse key signatures of depression in primary human neuronal cells.

FIGS. 2A-2G show data indicating that baricitinib reverses depressive behavior and associated inflammatory markers in a treatment resistant rat model. Baricitinib significantly reverses depressive behavior in a rat model of treatment resistant depression. These data reduce to practice that baricitinib reverses the behavior of depression and concomitantly increases non-depressive behavior in depressed rats. TNFa levels were measured by Meso Scale Discovery platform in the plasma from depressed mice treated intraperitonially (IP) with vehicle (saline) or baricitinib (25 mg). Immobility time (depression index-less immobile=LESS depressed; more mobility LESS depressed), time to immobility (longer time to immobility=LESS depressed) and active/climbing+swimming were evaluated and associated with TNFa levels.

FIG. 2A shows data indicating that baricitinib significantly reverses depression behavior in depressed rates versus depressed controls. Baricitinib significantly reverses depression behavior in depressed rates versus depressed controls at both physiological concentrations tested.

FIG. 2B shows data indicating that baricitinib significantly reverses depressed animal behavior to non-depressed behavior. Baricitinib also significantly reverses depressed animal behavior to non-depressed behavior at both physiological concentrations tested.

FIG. 2C shows data indicating that baricitinib significantly improves the short-term memory of depressed rats, to that of non-depressed rats.

FIG. 2D shows data indicating that baricitinib reverses treatment resistant depression by decreasing inflammation. Baricitinib reverts treatment resistant depression by decreasing inflammation (TNF-alpha).

FIG. 2E shows data indicating that baricitinib reverses treatment resistant depressive behavior as measured by immobility.

FIG. 2F shows data indicating that baricitinib reverses treatment resistant depressive behavior as measured by time to immobility.

FIG. 2G shows data indicating that baricitinib reverses treatment resistant depressive behavior as measured by active/climbing time.

FIGS. 3A-3I show data indicating that baricitinib significantly reduces immune activation and inflammation in vitro that mimics the depressive behavioral phenotype in humans, in an in vitro primary microglial model. Data indicates that baricitinib significantly reduces immune activation and inflammation in vitro that mimics the depressive behavioral phenotype in humans, as indicated by primary in vitro microglial model cellular markers of microglial activation; baricitinib significantly reduces overall activation induced by LPS, which mimics the immune dysregulation and inflammation that controls the depressive behavioral phenotype (h-gating strategy for activated cells).

FIG. 3A shows data indicating that baricitinib significantly reverses key markers of CNS inflammation and treatment resistant depression as measured by CD206.

FIG. 3B shows data indicating that baricitinib significantly reverses key markers of CNS inflammation and treatment resistant depression as measured by CD40.

FIG. 3C shows data indicating that baricitinib significantly reverses key markers of CNS inflammation and treatment resistant depression as measured by CD32.

FIG. 3D shows data indicating that baricitinib significantly reverses key markers of CNS inflammation and treatment resistant depression as measured by CD86.

FIG. 3E shows data indicating that baricitinib significantly reverses key markers of CNS inflammation and treatment resistant depression as measured by CD163.

FIG. 3F shows data indicating that baricitinib significantly reverses key markers of CNS inflammation and treatment resistant depression as measured by CD16.

FIG. 3G shows data indicating that baricitinib significantly reverses key markers of CNS inflammation and treatment resistant depression as measured by HLA-DR.

FIG. 3H shows the gating strategy to measure activated and non-activated microglia.

FIG. 3I shows data indicating that baricitinib significantly reverses the global population of activated microglia.

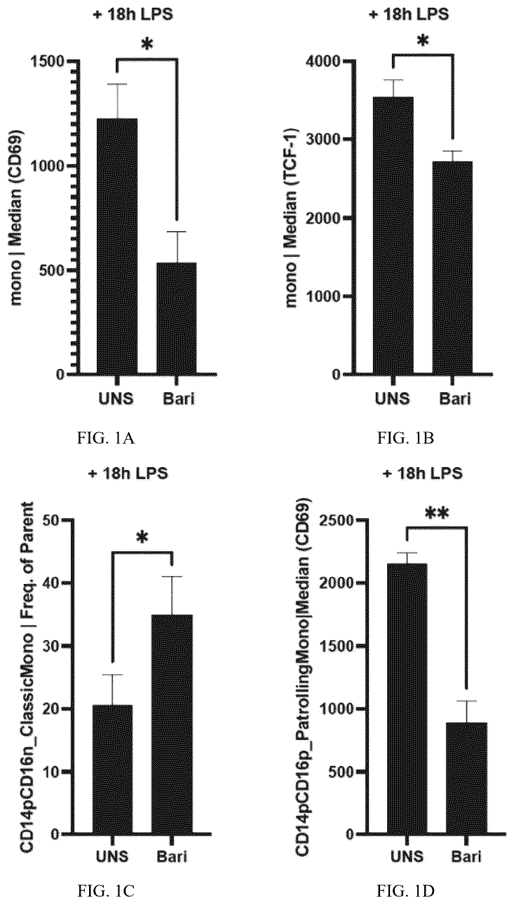

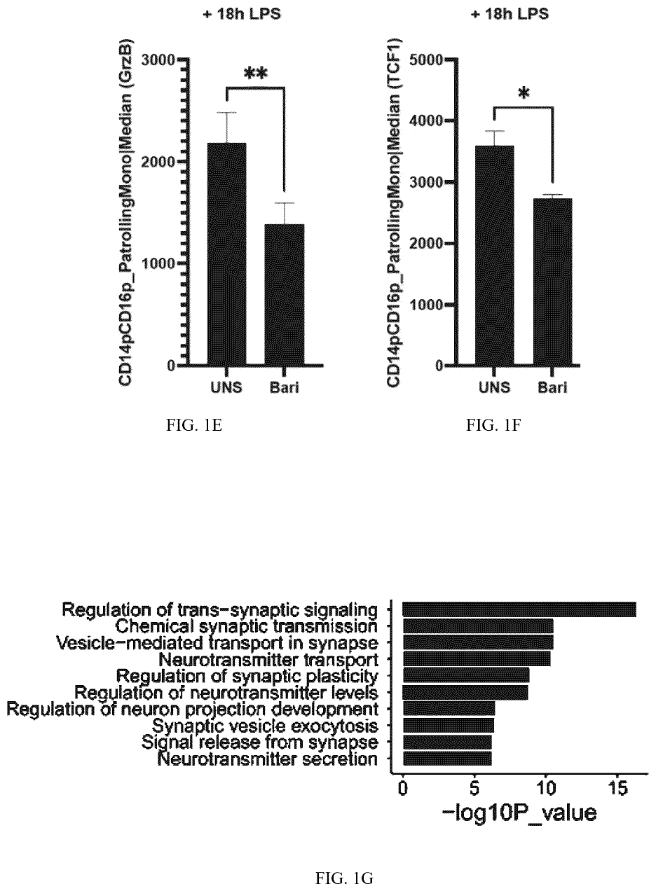

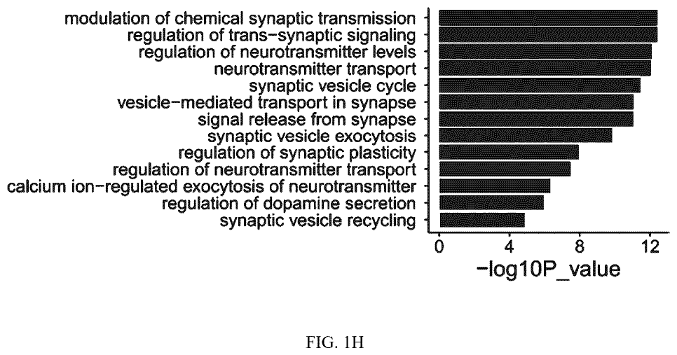

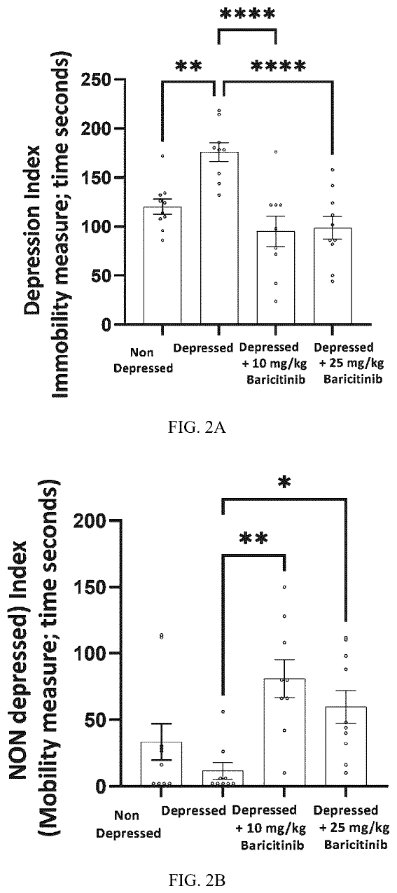

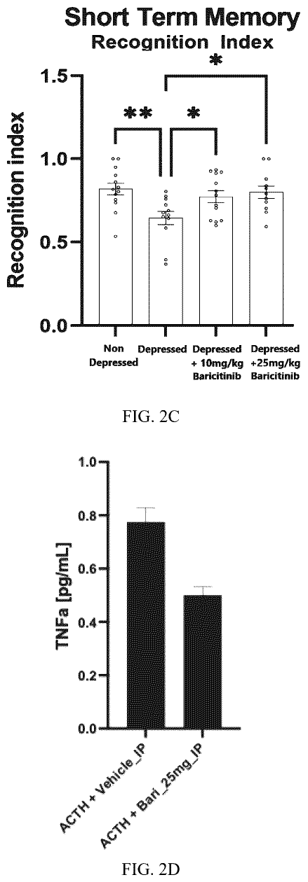

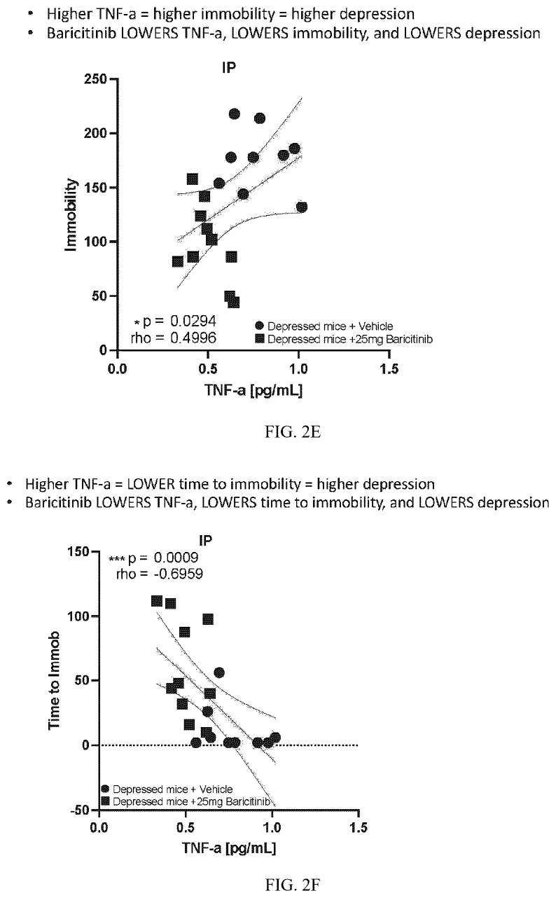

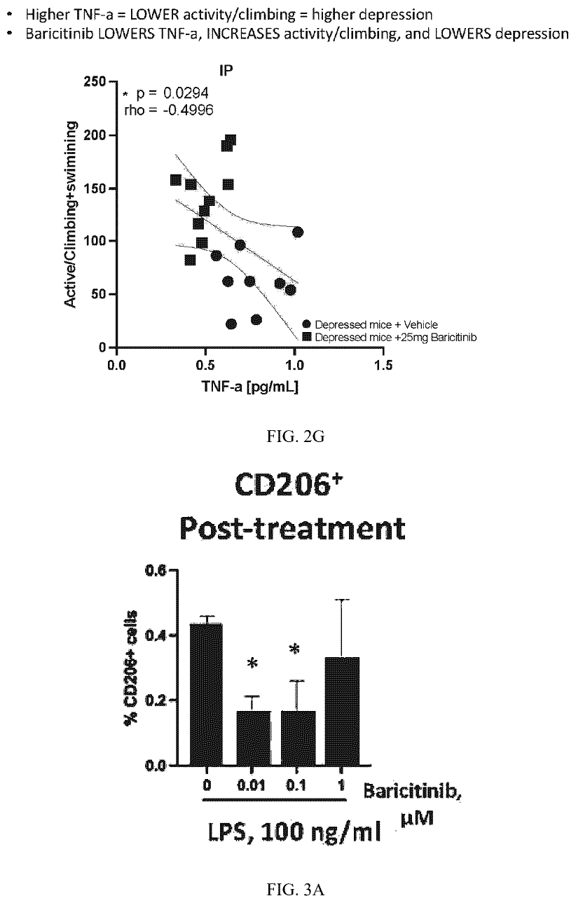

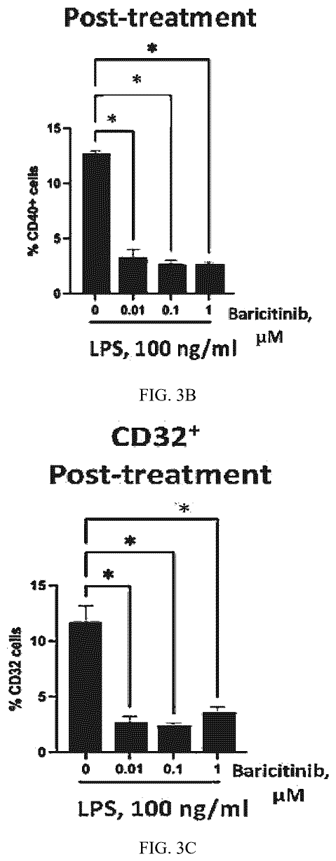

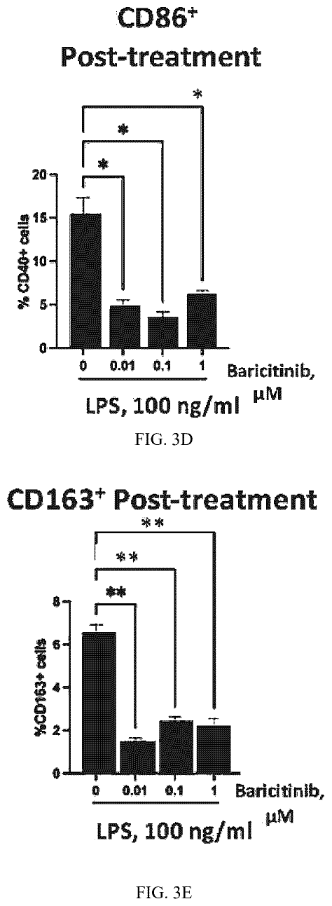

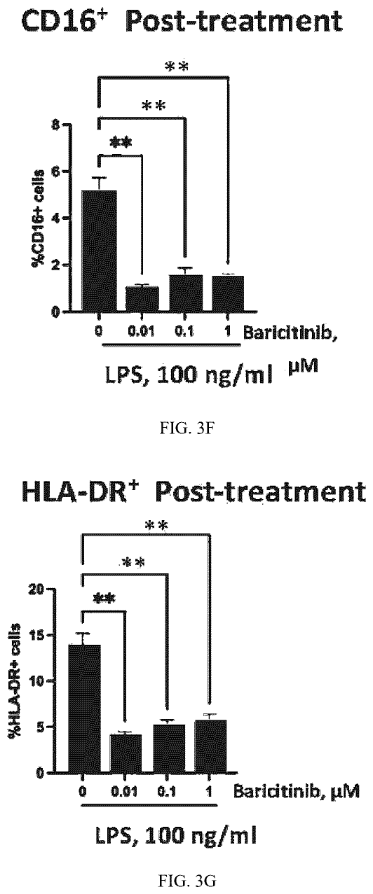

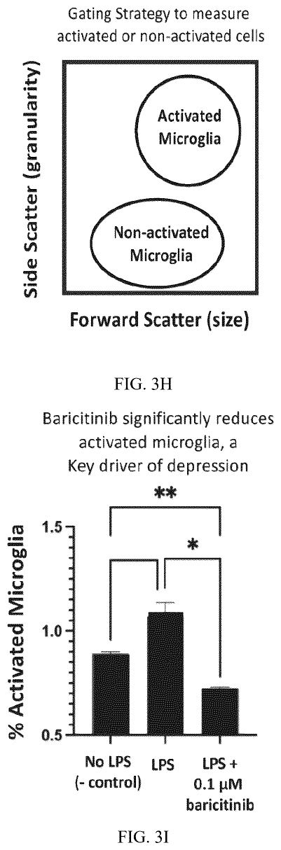

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “JAK inhibitor” or “Janus kinase inhibitor” refers to any variety of compounds, (small molecules, biological molecules, anti-JAK antibodies, interfering RNA, shRNA, nucleic acids/vectors encoding proteins, CRISPR-Cas mediated systems, etc.) that, when in contact with a Janus kinase enzyme (JAK1, JAK2, JAK3, TYK2) specifically bind with a Janus kinase enzyme altering the JAK-STAT signaling pathway. Examples include baricitinib, tofacitinib, upadacitinib cerdulatinib, gandotinib, lestaurtinib, momelotinib, pacritinib ruxolitinib, oclacitinib, peficitinib, fedratinib, filgotinib, delgocitinib, abrocitinib, all of which are contemplated for uses disclosed herein.

As used herein, the term “small molecule” refers to any variety of covalently bound molecules with a molecular weight of less than 900 or 1000 Da. Typically, the majority of atoms include carbon, hydrogen, oxygen, nitrogen, and to a lesser extent sulfur and/or a halogen. Examples include steroids, short peptides, mono or polycyclic aromatic or non-aromatic, heterocyclic compounds.

As used herein, “subject” refers to any animal, preferably a human patient.

As used herein, the terms “treat” and “treating” are not limited to the case where the subject (e.g. patient) is cured and the disease is eradicated. Rather, embodiments, of the present disclosure also contemplate treatment that merely reduces symptoms, reverses, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the term “combination with” when used to describe administration of an agent with an additional treatment means such that the agent may be administered prior to, together with, or after the additional treatment, or a combination thereof, such that multiple agents are available at some overlapping time.

The term “effective amount” refers to that amount of a compound or pharmaceutical composition described herein that is sufficient to effect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compound chosen, the dosing regimen to be followed, whether it is administered in combination with other agents, timing of administration, the tissue to which it is administered, and the physical delivery system in which it is carried.

The terms “drug,” “agent,” “pharmaceutical agent,” and similar terms are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect. Examples include chemotherapy agents, analgesics, steroidal anti-inflammatories, non-steroidal anti-inflammatoirenti-depressants, statins, antibiotics, anti-bacterial agents, anti-neoplastics, anti-spasmodics, modulators of cell-extracellular matrix interactions, proteins, hormones, enzymes and enzyme inhibitors, anticoagulants and/or antithrombic agents, and vasodilating agents.

The term “antidepressant” refers to a pharmacological agent which, when a therapeutically effective amount is present, the drug generally accepted as useful in the treatment of depression. Non-limiting examples include duloxetine, paroxetine, fluoxetine, levomilnacipran, escitalopram, citalopram, desvenlafaxine, doxepin, desipramine, imipramine, clomipramine, trimipramine, sertraline, trazodone, vilazodone, venlafaxine, amitriptyline, nortriptyline, protriptyline, isocarboxazid, phenelzine, tranylcypromine, bupropion, selegiline, mirtazapine, mianserin, and dextromethorphan.

The term “antipsychotic” refers to a pharmacological agent which, when a therapeutically effective amount is present, the drug generally accepted as useful in the treatment of psychosis, such as delusions (false beliefs) and hallucinations (secing or hearing something that is not there). Non-limiting examples include risperidone, quetiapine, olanzapine, ziprasidone, paliperidone, aripiprazole, clozapine, chlorpromazine, flupenthixol, fluphenazine, haloperidol, loxapine, perphenazine, pimozide, trifluoperazine, thiothixene and zuclopenthixol.

The terms “anti-inflammatory” and “anti-inflammatory agent” are also used interchangeably herein and mean and include a “pharmacological agent” and/or “active agent formulation” which, when a therapeutically effective amount is present the drug prevents or treats bodily tissue inflammation i.e., the protective tissue response to injury or destruction of tissues, which serves to destroy, dilute, or wall off both the injurious agent and the injured tissues. Examples include alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, etodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, and zomepirac sodium.

### Methods of Use

This disclosure relates to methods of treating, preventing, or reversing inflammation-associated central nervous system (CNS) disorders or conditions comprising administering an effective amount of a Janus kinases (JAK) inhibitor such as baricitinib to a patient in need thereof. In certain embodiments, the inflammation-associated CNS disorder is depression or treatment resistant depression and includes symptoms such as fatigue and cognitive dysfunction.

In certain embodiments, this disclosure relates to methods of treating inflammation associated depression comprising administering an effective amount of a JAK inhibitor such as baricitinib to a patient in need thereof. In certain embodiments, this disclosure relates to methods of treating depression comprising administering an effective amount of a JAK inhibitor such as baricitinib to a patient in need thereof. In certain embodiments, methods reported herein are contemplated to exclude patients having atopic dermatitis, rheumatoid arthritis, or alopecia.

In certain embodiments, the patient was administered an antidepressant and did not respond or becomes non-responsive to treatment. In certain embodiments, the patient was previously administered an antidepressant and continued to present with clinical depression. In certain embodiments, the patient was previously administered two different anti-depressants and continued to present with clinical depression. In certain embodiments, baricitinib is administered in combination with another antidepressant.

In certain embodiments, this disclosure relates to methods of treating inflammation-associated dopamine disruption to improve memory/cognition, motivation, motor activity, (treat or prevent) psychomotor slowing, prevent fatigue, improve anhedonia, improve memory, improve concentration, improve cognition, prevent (reduce) sleep disruptions, and prevent suicide comprising administering an effective amount of a Janus kinases (JAK) inhibitor such as baricitinib to a patient in need thereof.

In certain embodiments, the inflammation-associated CNS disorder or condition is schizophrenia. In certain embodiments, this disclosure relates to methods of treating or preventing schizophrenia comprising administering an effective amount of a Janus kinases (JAK) inhibitor such as baricitinib to a patient in need thereof. In certain embodiments, the JAK inhibitor is administered in combination with an antipsychotic.

In certain embodiments, the inflammation-associated CNS disorder or condition is Post Traumatic Stress Disorder (PTSD). In certain embodiments, this disclosure relates to methods of treating or preventing Post Traumatic Stress Disorder (PTSD) comprising administering an effective amount of a Janus kinases (JAK) inhibitor such as baricitinib to a patient in need thereof.

In certain embodiments, the inflammation-associated CNS disorder or condition is anxiety. In certain embodiments, this disclosure relates to methods of treating or preventing anxiety comprising administering an effective amount of a Janus kinases (JAK) inhibitor such as baricitinib to a patient in need thereof.

In certain embodiments, the inflammation-associated CNS disorder or condition is bipolar disorder. In certain embodiments, this disclosure relates to methods of treating or preventing bipolar disorder comprising administering an effective amount of a Janus kinases (JAK) inhibitor such as baricitinib to a patient in need thereof.

In certain embodiments, the inflammation-associated CNS disorder or condition is dementia. In certain embodiments, this disclosure relates to methods of treating or preventing dementia comprising administering an effective amount of a Janus kinases (JAK) inhibitor such as baricitinib to a patient in need thereof.

In certain embodiments, this disclosure relates to methods of improving memory comprising administering an effective amount of a Janus kinases (JAK) inhibitor such as baricitinib to a patient in need thereof.

In certain embodiments, this disclosure relates to methods of treating chronic fatigue syndrome comprising administering an effective amount of a Janus kinases (JAK) inhibitor such as baricitinib to a patient in need thereof.

In certain embodiments, this disclosure relates to methods of treating infection-related depression and depressive symptoms comprising administering an effective amount of a Janus kinases (JAK) inhibitor such as baricitinib to a patient in need thereof.

In certain embodiments, this disclosure relates to methods of treating virus-induced or related infection depression comprising administering an effective amount of a Janus kinases (JAK) inhibitor such as baricitinib to a patient in need thereof.

In certain embodiments, this disclosure relates to methods of treating HIV depression and depressive symptoms comprising administering an effective amount of a Janus kinases (JAK) inhibitor such as baricitinib to a patient in need thereof.

In certain embodiments, this disclosure relates to methods of treating bacterial-induced or related infection depression comprising administering an effective amount of a Janus kinases (JAK) inhibitor such as baricitinib to a patient in need thereof.

In certain embodiments, this disclosure relates to methods of treating depression caused by Lyme disease/post-treatment Lyme disease syndrome comprising administering an effective amount of a Janus kinases (JAK) inhibitor such as baricitinib to a patient in need thereof.

In certain embodiments, this disclosure relates to methods of treating or preventing chronic depression or acute depression comprising administering an effective amount of a Janus kinases (JAK) inhibitor such as baricitinib to a patient in need thereof. In certain embodiments, the subject is diagnosed with chronic depression which persistent depressive disorder or dysthymia, e.g., wherein symptoms must occur for at least one or two years.

In certain embodiments, this disclosure relates to methods of treating or preventing intense feelings of sadness, hopelessness, or emptiness, decreased speech, movement, thought speed, anxiety, restlessness, anhedonia, significant changes to sleep, eating habits, loss of energy, persistent fatigue, feelings of worthlessness, misplaced guilt, exaggerated guilt, fixating on past failures, social isolation, difficulty thinking, concentrating, making decisions, pain, headache, muscle pain, stomachache, suicidal thoughts, or suicidal actions comprising administering an effective amount of a Janus kinases (JAK) inhibitor such as baricitinib to a patient in need thereof.

In certain embodiments, this disclosure relates to methods of treating post-treatment Lyme disease syndrome comprising administering an effective amount of a Janus kinases (JAK) inhibitor such as baricitinib to a patient in need thereof.

In certain embodiments, this disclosure relates to methods of treating Jarisch-Herxheimer reaction comprising administering an effective amount of a Janus kinases (JAK) inhibitor such as baricitinib to a patient in need thereof. In certain embodiments, Jarisch-Herxheimer reaction is associated with syphilis, louse-borne relapsing fever, leptospirosis, and tick-borne relapsing fever.

In certain embodiments, this disclosure relates to methods of treating Lyme arthritis comprising administering an effective amount of a Janus kinases (JAK) inhibitor such as baricitinib to a patient in need thereof.

In certain embodiments, this disclosure relates to methods of treating neurologic Lyme disease/neuroborreliosis comprising administering an effective amount of a Janus kinases (JAK) inhibitor such as baricitinib to a patient in need thereof.

In certain embodiments, this disclosure relates to methods of treating acute CNS inflammatory conditions comprising administering an effective amount of a Janus kinases (JAK) inhibitor such as baricitinib to a patient in need thereof.

In certain embodiments, this disclosure relates to methods of treating stroke comprising administering an effective amount of a Janus kinases (JAK) inhibitor such as baricitinib to a patient in need thereof.

In certain embodiments, this disclosure relates to methods of treating traumatic brain injury (TBI) comprising administering an effective amount of a Janus kinases (JAK) inhibitor such as baricitinib to a patient in need thereof.

In certain embodiments, this disclosure relates to methods of treating spinal cord injury comprising administering an effective amount of a Janus kinases (JAK) inhibitor such as baricitinib to a patient in need thereof.

In certain embodiments, this disclosure relates to methods of treating rare and orphan inflammatory diseases comprising administering an effective amount of a Janus kinases (JAK) inhibitor such as baricitinib to a patient in need thereof.

In certain embodiments, this disclosure relates to methods of treating prion disease comprising administering an effective amount of a Janus kinases (JAK) inhibitor such as baricitinib to a patient in need thereof.

In certain embodiments, this disclosure relates to methods of treating inflammation-associated neuropsychiatric and neurodegenerative disorders and CNS pathologies comprising administering an effective amount of a Janus kinases (JAK) inhibitor such as baricitinib to a patient in need thereof.

In certain embodiments, the inflammation-associated CNS disorder or condition is Alzheimer's disease or Parkinson's disease.

In certain embodiments, for any of the methods disclosed herein the JAK inhibitor may be administered in combination with another drug, antidepressant, antipsychotic, or anti-inflammatory agent.

In certain embodiments, the JAK inhibitor is a small molecule. In certain embodiments, the JAK inhibitor is selected from baricitinib, abrocitinib, delgocitinib, fedratinib, filgotinib, oclacitinib, pacritinib, peficitinib, ruxolitinib, tofacitinib, and upadacitinib.

### Treatment or Reversal of Inflammation-Associated Depression, Treatment Resistant Depression, and Related Central Nervous System (CNS) Inflammatory Conditions With the JAK Inhibitor Baricitinib

Major Depressive Disorder has high prevalence, and many seek therapeutic interventions that fail to work in one third of depressed patients. Patients with treatment resistant depression (TRD) experience a chronic depression condition that fails to resolve after treatment with at least two antidepressants. Experiments indicate that inflammation strongly correlates with depression and TRD in humans and in animal models. Inflammation confers dysfunction in key mechanistic pathways that drive depression and TRD and related disorders, including but not limited to schizophrenia, Post Traumatic Stress Disorder, anxiety disorders, Alzheimer's Disease, Parkinson's Disease, and/or other neuropsychiatric and neurodegenerative disorders with CNS inflammatory pathologies.

This disclosure contemplates the use of JAK inhibitors such as baricitinib alone or in combination as a treatment, prevention, or reversal of disease in people living with depression, Treatment Resistant Depression and related disorders, including but not limited to schizophrenia, Post Traumatic Stress Disorder, anxiety disorders, Alzheimer's Disease, Parkinson's Disease, and or other neuropsychiatric and neurodegenerative disorders with CNS inflammatory pathologies.

In certain embodiments, a subject with depression or Treatment Resistant Depression has or is diagnosed with chronic inflammation that includes myeloid cell activation and impaired dopamine (DA) neurotransmission, motivational deficits including anhedonia, psychomotor deficits, fatigue and or other neuropsychiatric and neurodegenerative disorders.

In certain embodiments, a subject for treatment or prevention of a disease or condition reported herein has or is diagnosed with abnormal and/or elevated inflammatory markers such as C-reactive protein (CRP), interferon (IFN)-alpha or interleukin (IL)-6, decreased DA release in ventral striatum, e.g., association with anhedonia, psychomotor slowing, cognitive dysfunction, and fatigue.

In certain embodiments, a subject for treatment or prevention of a disease or condition reported herein has or is diagnosed with stress, inflammation, IL-6-associated striatal DA'ergic neuronal dysfunction, or inflammation-induced disruption of the blood-brain-barrier.

### Baricitinib in the Treatment of Depression and Treatment Resistant Depression

Experiments indicate that physiologically relevant concentrations of baricitinib can block key inflammatory and dysregulatory events in vitro that mimic the inflammatory dysregulation of depression and Treatment Resistant Depression observed in humans. These data were collected in primary human macrophages and microglia-like cells (Table 1), primary monocytes, and DA neurons derived from human induced pluripotent stem cells (hiPSCs) from healthy volunteers.

TABLE 1
Therapeutic concentrations of baricitinib demonstrate potency
for reversal of inflammation and immune dysregulation in
key CNS cells that contribute to Treatment Resistant Depression
and related CNS inflammatory pathologies.

| | | CD14/CD16/HLA- |
|---|---|---|
| CD11b⁺/CD163⁺ | CD163⁺/HLA-DR⁺ | DR⁺ |
| (Macrophages) | (Microglia-like cells) | Activated |
| EC₅₀, μM | EC₅₀, μM | Monocytes |
| 0.13 ± 0.04 | 0.09 ± 0.1 | 0.19 ± 0.07 |

Table 1 shows data indicating baricitinib significantly reduces inflammation in primary monocytes and myeloid cells, key drivers of inflammation in depression, Treatment Resistant Depression, and other inflammatory dysregulatory neuropathologies and symptoms. Table 1 summarizes effective concentration 50 (EC50) to block the neuropathological and inflammatory hallmarks of depression in microglial cells and activated monocytes.

These data indicate that baricitinib can be used towards the indication of inflammation-associated depression, Treatment Resistant Depression, and other CNS conditions including but not limited to schizophrenia, Post Traumatic Stress Disorder, anxiety disorders, Alzheimer's Disease, Parkinson's Disease, and/or other neuropsychiatric and neurodegenerative disorders with CNS inflammatory pathologies. This embodiment includes treatment or reversal of inflammation-associated depression, Treatment Resistant Depression and or related CNS pathologies with a Jak inhibitor such as baricitinib.

### Baricitinib Induces Reduced Depressive Behaviors Compared to Rats Without Treatment

In a behavioral model of rats with treatment resistant depression, rats treated with baricitinib have a significantly reduced depressive behaviors compared to rats without treatment. This indicates that baricitinib reverses depression. Measurement of immune dysregulated cytokines from this model indicate that baricitinib reduces these markers and correlate with reversal of depressive behavior. Microglial dysregulation drives depressive phenotype. In a human model of activated microglia, baricitinib reverses key markers that drive human depression. Dysfunction of dopaminergic neurons drive depression. In a primary human dopaminergic neuronal model, baricitinib reverses inflammatory dopaminergic phenotype and restores neurons to normal dopamine function. Measurements demonstrate that baricitinib reverses of depression behavior is associated with restoration of neuronal function and microglial activity.

### Primary Human-Derived Microglia Activation

Experiments were performed to determine the effect of baricitinib on primary human-derived microglia activation, which is a key driver of the depressive behavior phenotype. Microglia were derived from CD14 isolated monocytes from healthy donors and differentiated to microglia using a GM-CSF and IL-34 based media. LPS was added to mimic the immune dysregulation and inflammation that drives the CNS depressive behavior phenotype, including immune dysregulatory phenotype of depression as indicated by elevated microglial activation surface markers. Data demonstrate that baricitinib significantly reverses LPS-activated (depressive phenotype mimic) in primary human microglial cells.

### Baricitinib to Reverse Chronic Fatigue Syndrome

Interferon alpha drives chronic fatigue syndrome and baricitinib blocks these type 1 interferons; therefore, the use of baricitinib for the treatment of chronic fatigue syndrome is contemplated.

1. A method of improving motivation and reducing anxiety comprising administering an effective amount of a Janus kinases (JAK) inhibitor to a patient in need thereof.

2. The method of claim 1, wherein the patient exhibits symptoms of apathy.

3. The method of claim 1, wherein the patient exhibits symptoms of fatigue.

4. The method of claim 1, wherein the patient exhibits symptoms of cognitive dysfunction.

5. The method of claim 1, further comprising improving memory.

6. The method of claim 1, further comprising reducing decreased thought speed.

7. The method of claim 1, wherein the patient has feelings of social isolation.

8. The method of claim 1, wherein the patient exhibits symptoms of restlessness.

9. The method of claim 1, wherein the inhibitor is selected from baricitinib, abrocitinib, delgocitinib, fedratinib, filgotinib, oclacitinib, pacritinib, peficitinib, ruxolitinib, tofacitinib, and upadacitinib.

10. The method of claim 9, wherein the inhibitor is baricitinib.

11. A method of improving memory comprising administering an effective amount of a Janus kinases (JAK) inhibitor to a patient in need thereof.

12. The method of claim 11, further comprising improving concentration.

13. The method of claim 11, further comprising improving cognition.

14. The method of claim 11, wherein the inhibitor is selected from baricitinib, abrocitinib, delgocitinib, fedratinib, filgotinib, oclacitinib, pacritinib, peficitinib, ruxolitinib, tofacitinib, and upadacitinib.

15. The method of claim 14, wherein the inhibitor is baricitinib.

16. A method of treating dementia comprising administering an effective amount of a Janus kinases (JAK) inhibitor to a patient in need thereof.

17. The method of claim 16, wherein the inhibitor is selected from baricitinib, abrocitinib, delgocitinib, fedratinib, filgotinib, oclacitinib, pacritinib, peficitinib, ruxolitinib, tofacitinib, and upadacitinib.

18. The method of claim 17, wherein the inhibitor is baricitinib.
